(19) United States
(12) Patent Application Publication
(10) Pub. No.: US 2008/0031826 A1
(43) Pub. Date: Feb. 7, 2008

(54) Devices containing lipid emulsions

(75) Inventors: Peter Odegaard, Lysaker (NO); Svein-Erik Nilsen, Karlstad (SE); Magnus Hedman, Stathelle (NO)

Correspondence Address: Casimir Jones, S.C., 440 Science Drive, Suite 203, Madison, WI, 53711, US

(73) Assignee: Natural ASA, Lysaker (NO)

(21) Appl. No.: 11/800,370

(22) Filed: May 4, 2007

Related U.S. Application Data
(60) Provisional application No. 60/798,028, filed on May 5, 2006.

(51) Int. Cl. A61K 31/20; A61K 9/12; A61P 3/02; A61K 35/60

(52) U.S. Cl. 424/43; 514/560; 424/523; 514/547; 424/45

(57) ABSTRACT

The present invention relates to compositions and methods for the storage and consumption of a lipid emulsion. In particular, the present invention relates to the storage of an omega-3 lipid emulsion for consumption in a container under non-oxidizing conditions.

This application claims the benefit of U.S. Provisional Application 60/798,028, filed May 5, 2006, which is incorporated herein by reference in its entirety.

### FIELD OF THE INVENTION

The present invention relates to compositions and methods for the storage and consumption of a lipid emulsion. In particular, the present invention relates to the storage of an omega-3 lipid emulsion for consumption in a container under non-oxidizing conditions.

### BACKGROUND OF THE INVENTION

The foods we (e.g., humans) consume furnish four basic types of lipids; cholesterol, saturated fat, monounsaturated fat, and polyunsaturated fat. Eating too many foods rich in saturated fats has been associated with the development of degenerative diseases, including heart disease and cancer. Polyunsaturated fatty acids, however, are actually good for you and are increasing recognized as important to human health. Omega-3 fatty acids fall into this category, along with omega-6 fatty acids, another type of polyunsaturated fatty acid found in grains, most plant-based oils, poultry, and eggs. For reviews extolling the health benefits of omega-3 fatty acids see, for example, Engler, M M et al, 2006, J. Cardiovasc. Nurs. 21:17-24; MacLean C H et al., 2006, JAMA 295:403-15; Kang J X, 2005, World Rev. Nutr. Diet. 95:93-102; and Mickleborough T D, 2005, J. Asthma 42:3-50314, incorporated herein in their entireties. Omega-3 fatty acids are called essential fatty acids (EFAs) because they are critical for good health. However, the body cannot make them on its' own. For this reason, omega-3 fatty acids must be obtained from outside food sources. Key omega-3 fatty acids include eicosapentaenoic acid (EPA), docosahexanoic acid (DHA) and α-linolenic acid (ALA). EPA and DHA are found primarily in oily cold-water fish such as tuna, salmon, and mackerel. Aside from fresh seaweed, plant foods rarely contain EPA and DHA. ALA is found primarily in dark green leafy vegetables, flaxseed oils, and certain vegetable oils, and can be converted to EPA in the body.

Fresh fish consumption two to three times per week is a preferred method for obtaining adequate amounts of omega-3 fatty acids. However, many people find it difficult to eat that much fish per week, and mercury and other contaminants found in fresh fish are deterrents to consumption. Alternatively, capsules or bottles of liquid fish oil are available for consumption, but these alternatives are unpalatable, have a short shelf life as the fatty acids oxidize quickly, and an unmistakable fishy odor is ever-present.

As omega-3 fatty acids are essential for human consumption, what is required are novel modes of consumption, which are convenient and palatable, and a product that is able to be stored for long periods of time without oxidation.

### SUMMARY OF THE INVENTION

The present invention relates to compositions and methods for the storage and consumption of a dietary supplement, most preferably a lipid emulsion. In particular, the present invention relates to the storage of a dietary supplement, such as a lipid emulsion, for consumption in a container under non-oxidizing conditions. The present invention is not limited to use with any particular lipid or lipid emulsion. In some embodiments, the lipids contain omega-3 fatty acids or conjugated linoleic acid or combinations thereof. In some embodiments, the lipids are provided as triglycerides, diglycerides, fatty acids, ethyl- or methyl esters or phsopholipids.

The present invention furnishes omega-3 fatty acids in a palatable and convenient emulsion. Omega-3 fatty acids and conjugated linoleic acids are easily oxidizable, and the present invention furnishes a system where the lipid is protected from oxidation. To protect the lipid during consumption, the emulsion is provided in a sealed bag in a container, external to which is an inert gas (e.g., nitrogen, etc.) that serves as a propellant for emptying the bag containing the lipid emulsion. This system serves to protect the lipid from oxidation while in the container. The consumer dispenses the needed amount of emulsion and the remainder is kept air tight in the container until all used up.

Accordingly, in some embodiments, the present invention provides a device comprising a container housing a dietary supplement and an inert gas, wherein said inert gas pressurizes said container to allow delivery of said dietary supplement. In some embodiments, the dietary supplement comprises a mixture of natural fruits and lipids. In some embodiments, the lipids are unstable to oxidation. In some embodiments, the unstable lipid comprises an essential fatty acid. In some embodiments, the essential fatty acid is an omega-3 fatty acid. In some embodiments, the essential fatty acid is gamma-linolenic acid. In some embodiments, the dietary supplement is conjugated linoleic acid. In some embodiments, the lipid is krill oil. In some embodiments, the dietary supplement further comprises an emulsifier. In some embodiments, the inert gas is nitrogen. In some embodiments, the dietary supplement is further housed within a sealed bag within said container. In some embodiments, the lipids are selected from the group consisting of fish oil, krill oil, and phospholipids comprising omega-3 fatty acids.

In some embodiments, the present invention further provides a composition comprising an emulsion of natural fruit concentrates, an emulsifier, an omega-3 fatty acid, and a sugar substitute.

In some embodiments, the present invention provides methods of using the foregoing device. In some embodiments, the present invention provides methods of providing an effective daily amount of a lipid dietary supplement comprising pressurizing an emulsion comprising a lipid dietary supplement in a container comprising a nozzle so that an effective daily amount can be delivered via said nozzle. In some embodiments, the effective daily amount of the lipid dietary supplement is from about 200 mg to about 10 grams of said lipid, preferably from about 500 mg to about 3 grams of said lipid. In other embodiments, the present invention provides method of providing an oxidation-stabilized lipid that is normally unstable to oxidation comprising pressurizing an emulsion comprising a lipid that is normally unstable to oxidation in a container with an inert gas so that said lipid is stable to oxidation, wherein said container comprises a nozzle for delivery of said emulsion.

### DESCRIPTION OF THE FIGURES

FIG. 1 shows a view of an embodiment of the pouch which is integrated with a mounting cup for mounting on the neck of the container.

FIG. 2 shows a cross-sectional view of an embodiment of the container and pouch.

FIG. 3 shows a cross-sectional view of an embodiment of a dispensing container.

### DEFINITIONS

As used herein, the term “emulsion” refers to a mixture of two immiscible (e.g., unblendable) substances, wherein one substance (the dispersed phase) is dispersed in the other (the continuous phase).

As used herein, the term “inert gas” refers to any gas that is not reactive under normal circumstances. The inert gas can be either molecular (e.g., noble gas) and/or elemental in nature. For example, in one embodiment of the present invention nitrogen is utilized as an inert gas.

As used herein, the term “lipid” to a class of hydrocarbon-containing organic compounds essential for the structure and function of living cells. Lipids are characterized by being water-insoluble. A preferred embodiment of the present invention is the use of the omega-3 fatty acid (e.g., lipid) in an emulsion for human consumption.

### DETAILED DESCRIPTION OF THE INVENTION

The lipid emulsion of the present invention in provided in a container, which is exemplified by the accompanying drawings (U.S. Pat. No. 6,547,770 B2, incorporated herein in its entirety), in which preferred embodiments of the invention are shown. The container may, however, take the shape of many different forms and should not be construed as limited to the specific embodiments set forth herein. These embodiments are provided as exemplary models and those skilled in the art will appreciate the many shapes a container can take, thereby conveying the scope of the present invention.

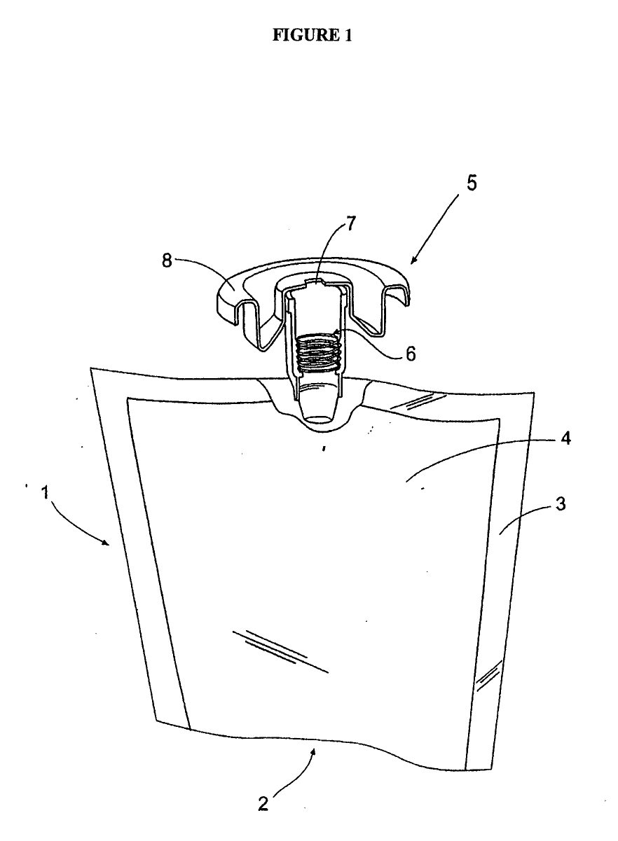

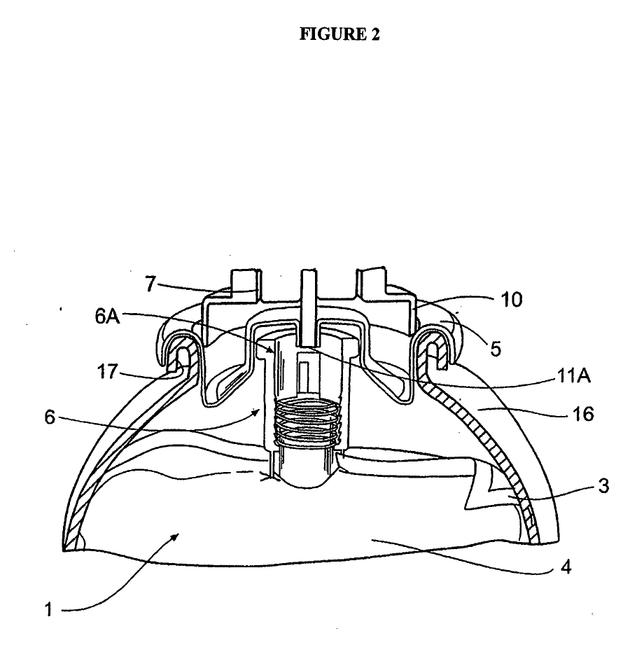

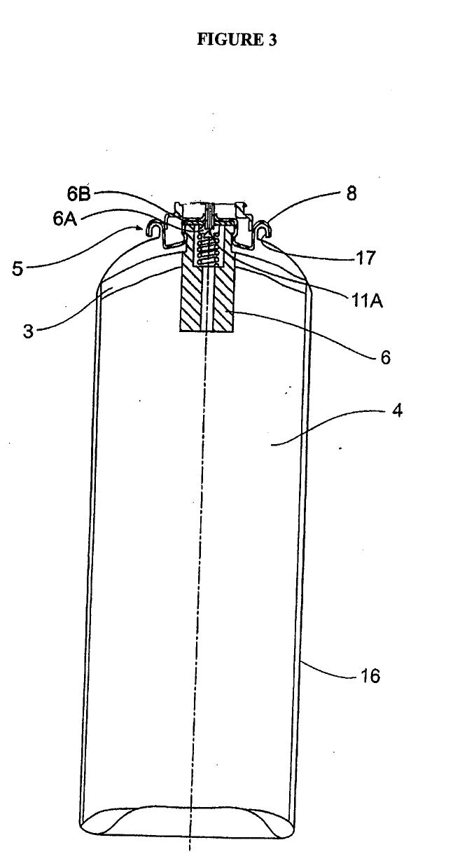

FIG. 1 illustrates a pouch (1) to be accommodated inside a pressurized container. The pouch comprises a bag (4) that is flat in its unfilled state, and which is formed by an essentially diffusion proof multilayered laminate. The volume of the bag is 30-400 ml of, the two most preferred volumes being 75 ml and 200 ml. At the lower end (2) of the bag there is a folded, preferably double folded, edge. Along the longitudinal edges of the bag, there are sealing edges (3), normally consisting of welded edges. These sealed edges exhibit a certain width, which will result in a fictional securing of the bag inside the container. At the upper end of the bag there is arranged a mounting cup (5) that is connected, preferably welded, to the bag at a connection member (6). In this regard, the bag should not be too big, in order for the bag not to let go from the connection member if the device is dropped onto the floor. As can be seen in FIG. 2, the connection member consists of a plastic tube, accommodating a conventional non-return valve (6A) including a spring-valve body-valve seat assembly which opens up when the valve body is forced out of contact with the valve seat (6B) counteracting the resilient force of the spring. The exit of the connection member for the lipid emulsion in the bag is a small hole in the mounting cup. As seen on FIG. 3, the top part of the mounting cup consists of a metal cup (8) provided with a double walked circumference below which there is arranged a gasket. The shape of the container (16) is exemplary only, and multiple container shapes are contemplated. Additionally, multiple container compositions (e.g., metal, paper, plastic, etc.) are contemplated. The lipid emulsion exits the container by pressure exerted by an inert gas when a nozzle is activated. The activation of the nozzle allows for the gas to exert pressure on the bag containing the lipid emulsion, thereby expelling the lipid emulsion from the container. Multiple nozzle configurations are contemplated, and the present invention is not limited to any particular nozzle configuration. In a preferred embodiment, the spray mechanism provides a conical spray pattern, where spray angle alpha can be set to 0 degrees.

In one embodiment, the present invention is designed to be a “bag in the can” concept to achieve a pressurized dispensing device without the use of halogenated compounds in the drive gas (e.g., freon). In some embodiments, the “bag in the can” concept includes a pressure container having a closed bottom and an open top defining a neck, for accommodating a pressurized and sealed flexible pouch. In some embodiments, the pouch, which is accommodated inside the container, is made of an essentially diffusion-proof barrier material and exhibits a valve that is integrated with a mounting cap adapted to fit the neck of the container. In some embodiments, when the container is to be filled with liquid and inert gas (e.g., nitrogen), the inert gas is filled into the container first. In some embodiments, the open neck of the container is sealed by the mounting cup being crimped onto the neck of the container. In some embodiments, the liquid is filled into the pouch via the valve in the mounting cup, so that a desired total pressure is achieved inside the pouch/container. This type of container, for example, allows for the inert gas and the lipid emulsion to never come into contact with each other.

In one embodiment, the propellant used in the lipid emulsion-containing container of the present invention is an inert gas. In some embodiments, nitrogen is the inert gas. The present invention is not limited by the inert gas used, and other inert gases are contemplated (e.g., argon, helium, neon, xenon, for example).

In one embodiment, the present invention is a composition comprising an oil-in-water emulsion of natural fruit concentrates, a high quality fish oil, an emulsifier, a citrus juice, a sugar or sugar substitute, and water. In some embodiments, the fruit concentrate comprises orange juice and banana. However, the present invention is not limited by the type of fruit concentrate used, and many combinations of fruit concentrates are contemplated. In some embodiments, the emulsion comprises at least 10% of the fruit concentrate, preferably at least 20% o the fruit concentrate, more preferably at least 50% of the fruit concentrate.

In one embodiment, the emulsion comprises at least 10% of a lipid (i.e., oil for the oil-in-water emulsion), preferably at least 20% of a lipid, more preferably at least 25% of a lipid. The present invention is not limited to the use of any particular lipid. In some embodiments, the lipids are unstable to oxidation. In some embodiments, the lipids are krill oil, fish oil, or phospholipids or triglycerides or mixtures thereof comprising omega-3 fatty acids. In some embodiments, the lipids comprise fatty acids, and in particularly preferred embodiments, essential fatty acids. The present invention is not limited to the use of any particular fatty acids. Suitable essential fatty acids include, but are not limited to, ω3 fatty acids as 9,12,15-octadecatrienoate (gamma-linoleic acid (GLA)); 6,9,12,15-octadecatetraenoate; 11,14,17-eicosatrienoate; 8,11,14,17-eicosatetraenoate; 5,8,11,14,17-eicosapentaenoate; 7,10,13,16,19-docosapentaenoate; and 4,7,10,13,16,19-docosahexaenoate; ω6 fatty acids such as 6,9,12-octadecatrienoate; 8,11,14-eicosatrienoate; 5,8,11,14-eicosatetraenoate; 7,10,13,16-docosatetraenoate and 4,7,10,13,16-docosapentaenoate; ω9 fatty acids such as 6,9-octadecadienoate; 8,11-eicosadienoate; and 5,8,11-eicosatrienoate; conjugated linoleic acid in any of its isomeric forms (e.g., 9,11-, 10,12,8,10-, or 11,13-CLA in any of the cis, trans cofigurations such as t10,c12-, c9,t11-, etc.); medium chain fatty acids such as decanoic acid, undecanoic acid, 10-undecanoic acid, lauric acid, cis-5-dodecanoic acid, tridecanoic acid, myristic acid, myristoleic acid; and long chain fatty acids such as pentadecanoic acid, palmitic acid, palmitoleic acid, heptadecanoic acid, stearic acid, elaidic acid, oleic acid, nonadecanoic acid, eicosanoic acid, cis-11-eicosenoic acid, 11,14-eicosadienoic acid, heneicosanoic acid, docosanoic acid, erucic acid, tricosanoic acid, tetracosanoic acid, nervonic acid, pentacosanoic acid, hexacosanoic acid, heptacosanoic acid, octacosanoic acid, nonacosanoic acid, triacosanoic acid, vaccenic acid, tariric acid, and ricinoleic acid.

In some embodiments, the percentage of omega-3 fatty acids in the lipid is at least 20% of the lipid, more preferably at least 30% of the lipid. In some embodiments, the lipid is an oil from a marine organism. The present invention is not limited to the use of particular oil from a marine organism. Indeed, many sources of oil are contemplated and include, for example, krill, salmon, tuna, mackerel, halibut, and other oils.

In one embodiment, the present invention further comprises a citrus juice. In some embodiments, the citrus juice is selected from a list consisting of lime, lemon, orange, and grapefruit. In some embodiments, the citrus juice used can be from a single source, or a mixture of several juices. In some embodiments, the amount of citrus juice in the emulsion is at least 1% of the total emulsion. In some embodiments, the amount of citrus juice is at least 2%, preferably 3%, more preferably at least 5.5% of the total amount of the emulsion.

In one embodiment, the present invention further comprises a sugar or sugar substitute. In some embodiments, the sugar is of plant origin. In some embodiments, the sugar is a sugar substitute. In some embodiments, the sugar or sugar substitute is selected from a list consisting of sucrose, fructose, glucose, aspartame, and xylitol. In some embodiments, the sugar is preferably fructose. In some embodiments, the amount of fructose in the lipid emulsion is at least 0.5%, preferably at least 1.0%, more preferably at least 2.0%, most preferably at least 2.6% of the lipid emulsion.

In one embodiment, the present invention further comprises an emulsifier. In some embodiments, the emulsifier is lecithin (e.g., phosphatidylcholine). In some embodiments, lecithin is derived from soybeans. However, lecithin is also found in egg yolks and is a viable source within the scope of the present invention. In some embodiments, the amount of emulsifier in the lipid emulsion is at least 1%, preferably at least 2%, more preferably at least 3% of the lipid emulsion. For example, the amount of emulsifier used is dependent on the degree of emulsion desired in each preparation of the present invention. The present invention is not limited by the emulsifier used, indeed other emulsifiers known in the art are equally applicable to the present invention. Exemplary methods for creating emulsions can be found, for example, in Food Emulsions, 4^{th} Edition, 2003, S E Friberg et al. Eds., p. 900, incorporated herein in its entirety.

In some embodiments, the present invention further comprises a viscosity modifier. The present invention is not limited to the use of any particular viscosity modifier. Indeed, the of a variety of viscosity modifiers is contemplated, including, but not limited to xanthan gum, guar gum, carrageenan (kappa, iota, or lambda), locust bean gum, pectin, gellan, konac flour, microcrystalline cellulose, and starches such as corn, tapioca, potato or rice starch. The foregoing ingredients may be used alone or in combination.

### EXPERIMENTAL

### Example 1

Two different lipids have been packaged using the containers of the present invention, involving various equipment from APV Innvensys, Denmark.

Machinery:
Flex-Mix™ Processor (Aceptic)
   +250 litre pressure vessel, 100% vacuum
   +Mixing agitator with scraper blades
   +Pump arrangement, control panel with datalogger
Scraped surface heat exchanger, aceptic
   +Variable speed feed pump, 100 to 500 liter per hour
   +Back pressure pump
   +Mass flow meter
   +Control panel with datalogger
One stage Rannie homogenizer machine 800 bar
Intasept 2232 aceptic filler
   +Compact filler can filler for bag-in-box filling

Procedure: The Flex-Mix™ Processor, was initially put under full vacuum, then flushed with dry pure nitrogen and re-establishing the full vacuum. The lipid was added by suction and fully protected from oxygen and then through same tube, the lecithin was also added. The machine was left for mixing for a few minutes to let the lecithin have enough time for interacting with the lipid phase. All juice concentrates except the lime juice was added to mixing vessel by suction through the same tube, flushing remaining lecithin with it. The machine was again left for a few minutes to start the emulsifying process, thereby further protecting the lipid.

A premix of fructose, potassium sorbate, lime juice and water was premixed through a high shear mixer and added through a hopper. Again the machine was left for a few minutes for good mixing of materials.

Finally, the pressure in the machine was gradually increased to atmospheric pressure by adding nitrogen. The agitator was stopped, the mixer set to 40% and circulation pump set to 30%. The main manhole to machine was opened and xantangum and aroma ingredient was added. Immediately the manhole was closed again and vacuum reapplied. This procedure has been shown to give the minimum entrapment of gas bubbles in the emulsion.

The agitator was turned on again to 20% for 5 minutes while leaving the mixer setting and circulation setting untouched. After turning agitator off again, the machine was left for 30 min for premixing.

Samples were pulled from premixer and sent to microscopy testing, to ensure good premixing and minimum entrapment of gas. After satisfactory results, the product was sent to the scraped surface heat exchanger.

Product was heated from about 30 deg C. to 95 deg C. for 45 sec, then cooled to 35 deg C., before being sent to homogenizer.

All material was passed through the one stage Rannie homogenizer for its final processing. The lipid particle size was first controlled by standard microscopy method and later verified by analyses for their particle size distribution by Helos Sympatec (laser diffraction, volume weighted distributions, 0.1-35 μm, a few analyses also in the range from 0.5-87.5 μm). A few selected samples are analysed for their viscosity by means of a Brookfield Viscometer, all to confirm consistency in the samples.

Final stage was filling of product in 200 kg aceptic bag-in-box system using the Intasept 2232. Samples for later quality testing was filled in 10 kg bags.

Several formulas with two lipid types have been made the same way.

GC Rieber 18/12 Special Food Grade (Omega-3)

| Raw material # Name | Formula % | Weight (kg) |
|---|---|---|
| Orange juice cons. 65% Brix | 40.5 | 162 |
| Banana juice cons. 72% Brix | 22 | 88 |
| GC RIEBER 18/12 SFG | 26 | 104 |
| Sternfild E60 lecitin | 2.9 | 11.6 |
| Xanthan gum | 0.2 | 0.8 |
| Orange Aroma oil | 0.03 | 0.12 |
| fructose | 2 | 8 |
| Lime juice cons. 65% Brix | 1.5 | 6 |
| Sorbat | 0.1 | 0.4 |
| Water | 5 | 20 |
| Total | 100.23 | 400 |

This formula was split in two for running on the Flex-Mix™ Processor.

Tonalin CLA 80 Triglyceride delivered from Cognis

| Raw material# Name | Formula % | Weight (kg) |
|---|---|---|
| Orange juice cons. 65% Brix | 40.5 | 101.25 |
| Banana juice cons. 72% Brix | 22 | 55 |
| Tonalin TG 80 | 29.5 | 73.75 |
| Sternfild E60 lecitin | 2.9 | 7.25 |
| Xanthan gum | 0.15 | 0.375 |
| Orange Aroma oil | 0.03 | 0.075 |
| fructose | 2 | 5 |
| Lime juice cons. 65% Brix | 1.5 | 3.75 |
| Sorbat | 0.1 | 0.25 |
| Water | 1.5 | 3.75 |
| Total | 100.18 | 250 |

We claim:

1. A device comprising a container housing a dietary supplement and an inert gas, wherein said inert gas pressurizes said container to allow delivery of said dietary supplement.

2. The device of claim 1, wherein said dietary supplement comprises a mixture of natural fruits and lipids.

3. The device of claim 2, wherein said lipids are unstable to oxidation.

4. The device of claim 3, wherein said unstable lipid comprises an essential fatty acid.

5. The device of claim 4, wherein said essential fatty acid is an omega-3 fatty acid.

6. The device of claim 4, wherein said essential fatty acid is gamma-linolenic acid.

7. The device of claim 1, wherein said dietary supplement is conjugated linoleic acid.

8. The device of claim 1, wherein said dietary supplement is krill oil.

9. The device of claim 1, further comprising an emulsifier.

10. The device of claim 1, wherein said inert gas is nitrogen.

11. The device of claim 1, wherein said mixture is further housed within a sealed bag within said container.

12. The device of claim 2, wherein said lipids are selected from the group consisting of fish oil, krill oil, and phospholipids comprising omega-3 fatty acids.

13. A composition comprising an emulsion of natural fruit concentrates, an emulsifier, an omega-3 fatty acid, and a sugar substitute.

14. A method of providing an effective daily amount of a lipid dietary supplement comprising pressurizing an emulsion comprising a lipid dietary supplement in a container comprising a nozzle so that an effective daily amount can be delivered via said nozzle.

15. A method of providing an oxidation-stabilized lipid that is normally unstable to oxidation comprising:
pressurizing an emulsion comprising a lipid that is normally unstable to oxidation in a container with an inert gas so that said lipid is stable to oxidation, wherein said container comprises a nozzle for delivery of said emulsion.
